(19) United States
(12) Patent Application Publication
(10) Pub. No.: US 2004/0096972 A1
(43) Pub. Date: May 20, 2004

(54) Chimeric plasmid comprising a replicative retroviral genome, and uses

(75) Inventors: Muriel Audit, Paris (FR); Francois-Loic Cosset, Lyon (FR)

Correspondence Address: HESLIN ROTHENBERG FARLEY & MESITI PC, 5 COLUMBIA CIRCLE, ALBANY, NY, 12203, US

(73) Assignees: GENETHON; INSTITUT NATIONAL DE LA SANTE ET DE LA RECHERCHE MEDICALE, Paris (FR)

(21) Appl. No.: 10/677,558

(22) Filed: Oct. 2, 2003

Related U.S. Application Data
(63) Continuation of application No. PCT/FR02/03934, filed on Nov. 18, 2002.

(30) Foreign Application Priority Data
Nov. 20, 2001 (FR) 01.14976

(51) Int. Cl. C12N 7/01; C12N 15/86

(52) U.S. Cl. 435/456; 435/235.1; 435/320.1

(57) ABSTRACT

Disclosed is a plasmid comprising a replicative retroviral genome, characterized in that it contains a psi (ψ) sequence, gag and pol sequences originated from the genome of an MLV virus, and a chimeric env sequence. The chimeric env sequence comprises a region corresponding to part of the envelope originating from the genome of an MLV virus and a region corresponding to part of the envelope originating from the genome of a GaLV virus.

### CROSS-REFERENCE TO RELATED APPLICATIONS

This application is a continuation of PCT International Application No. PCT/FRO2/03934 filed Nov. 18, 2002 and published on May 30, 2003 as WO 03/044202 which claims priority of French Application No. 01.14976 filed Nov. 20, 2001. The entire disclosures of the prior applications are incorporated herein by reference.

### REFERENCE TO SEQUENCE LISTING

This application includes a “Sequence Listing” provided in paper and computer readable form, the entire contents of which are incorporated herein by reference.

### FIELD OF THE INVENTION

The invention relates to a chimeric plasmid comprising a replicative retroviral genome containing gag, pol and env nucleotide sequences originating from retroviruses from distinct species. It also relates to a retrovirus of the MLV (Murine Leukemia Virus) type and of the GaLV (Gibbon Ape Leukemia Virus) type produced by a cell line expressing said plasmid. It also relates to a bacterium producing the plasmid. Furthermore, it relates to a virion containing a replicative retroviral genome originating from retroviruses from distinct species. The invention also relates to use of the virion as a positive control in a test intended to detect the replicative capacity of a retroviral vector of the MLV GaLV type, in particular a mobilization test. Finally, the invention relates to a kit for carrying out said test.

### BACKGROUND OF THE INVENTION

The main aim of gene therapy is to introduce, in vitro, in vivo or ex vivo, a gene of interest into cells in order to produce, for example, a recombinant protein or peptide. The introduction of the gene of interest into the cell is carried out via either viral vectors (in practice AAVs, adenoviruses or retroviruses, etc.) or synthetic vectors (in particular, synthetic lipid, etc.), into the structure of which is inserted the gene of interest. The present invention relates exclusively to the field of gene therapy using retroviral vectors.

All retroviral genomes have the same basic structure, including in particular the gag, pol and env genes. The gag gene encodes the structural proteins (capsid, matrix and nucleocapsid), the pol gene encodes the enzyme functions, while the env gene encodes the envelope proteins. Each end of the genome exhibits long terminal repeats (LTRs) which contribute to the replication, to the integration in the cell and the expression of the viral genome, and also a Ψ (PSI) sequence responsible for encapsidation of the retroviral genome into the protein envelope.

One of the essential conditions for it to be possible to use the retroviral vector in gene therapy is that it exhibits no ability to replicate. In fact, replication-competent retroviruses (RCRs) can induce a massive invasion of the organism and, subsequently, various pathological conditions. The studies by Donahue (1) have shown that RCRs which have been generated during the production of retroviral vectors induce leukemias in immunodepressed rhesus monkeys.

The 1^{st} generation of MLV-derived retroviral vectors was made up in the following way: the gene to be introduced into the target cells was placed in a transfer vector comprising in particular LTRs (Long Terminal Repeats) and the ψ sequence of the MLV virus. This vector was introduced into a “packaging” cell expressing, from a single molecular construct in which the ψ sequence had been deleted, the gag, pol and env genes of MLV. A certain number of packaging lines on this model were proposed, such as, for example, the ψ2, ψ-Am and PA 12 lines. However, it was shown that RCRs could be generated in such packaging lines subsequent to recombinations between the transfer vector (containing the gene of interest) and the viral sequences of the packaging line (molecular construct allowing expression of the gag, pol and env viral genes) (2).

Other models of MLV packaging lines were proposed, in which, besides the absence of the encapsidation signal ψ, the LTRs were partially or totally deleted. The aim of these additional deletions was to decrease the probability of reconstitution, by recombinations, of a retroviral genome capable of producing RCRs. Such lines correspond, for example, to the PA 317 line. However, it was shown that these lines were, like the previous ones, capable of generating RCRs (3).

To decrease the probability of the recombinations possibly occurring between the transfer vector and the vector of the packaging line, further modifications were introduced into the production of MLV vectors. The viral sequences of the packaging line were placed on two different vectors instead of a single one (additional recombinations were then necessary in order for RCRs to emerge). In practice, the major modification introduced compared to the packaging lines of the PA 317 type is the following: the gag and pol genes are introduced via a first plasmid and the env gene by a second plasmid. Such lines correspond, for example, to the GP+Env Am12 line (4).

Given the remaining risk of recombinations between the viral sequences of the packaging lines and the retroviral transfer vectors, and the possible consequences of such recombinations, searching for RCRs in preparations of MLV vectors was made obligatory by the FDA (Food and Drug Administration) for batches of MLV vectors intended for clinical trials (5). The tests required by the FDA must be carried out on the vector-producing cells, on their supernatant, and also on the patients who have undergone the gene therapy.

Various methods can be used to detect RCRs. The most commonly used to test the vector-producing cells and the viral supernatants is a mobilization test. This test consists in incubating the sample to be tested with a mobilizing line which is permissive to infection with the vector produced by the cells or contained in the supernatant. The mobilizing line carries a vector comprising in particular the LTRs and the Ψ sequence of MLV and also a gene for resistance to an antibiotic. This mobilization vector is a sort of trap. When the mobilizing cells are infected with RCRs, these RCRs provide the viral proteins required for the production of infectious particles and these particles integrate equally their own genome or the mobilization vector. The supernatant from the mobilization cells is then transferred onto indicator cells which are treated with the antibiotic corresponding to the resistance gene carried by the mobilization vector. The existence of indicator cells resistant to the antibiotic indicates the presence of RCRs in the supernatant or the producer cells tested.

The document by MILLER (6) describes a plasmid called “pAM”, the expression product of which (the virus) is used as a positive control for searching for RCRs in preparations of retroviral vectors of the amphotropic MLV type. This virus can be used as a control because it is similar to the RCRs which can be generated in preparations of amphotropic MLV vectors. The use of this positive control has at this time been made obligatory by the FDA. It is marketed by the ATCC (American Type Culture Collection) in the form of a viral supernatant (ref. VR-1450) and in the form of a cell line NIH3T3, producing this virus (ref. VR-1448). The plasmid pAM was obtained by replacing the ecotropic env gene of Moloney MLV with the amphotropic env gene of MLV 4070A. The sequence of pAM is listed in GENBANK under the accession number AF 010170. This sequence comprises in particular a cloning vector, pBR322, and the genome of the virus made up in the following way: 5′ LTR (bases 145 to 736), gag gene (bases 1212 to 2828), pol gene (bases 2829 to 6428), env gene (bases 6368 to 8332) and 3′ LTR (bases 8374 to 8967).

The molecular construction of pAM was relatively easy due to the strong sequence homologies and functional homologies of the genomes of the two MLVs used to prepare this construct. Specifically, due to the presence of allelic restriction sites (same restriction sites at the same places in the 2 genomes considered), this construct was prepared by simple enzyme digestion of the recipient plasmid and of the donor plasmid, followed by ligation of the 2 fragments to be combined.

The most commonly used MLV-derived retroviral vectors are those which carry an envelope allowing infection of human cells, for example an amphotropic envelope (derived from the mouse virus MLV) or a GaLV envelope (derived from a monkey virus). The use of a GaLV envelope rather than an amphotropic envelope has various advantages. First, the GaLV envelope allows infection of more human cell types than the amphotropic envelope (7). Second, the use of vectors carrying structural genes and an envelope gene derived from different species, as is the case for the MLV-GaLV vectors, decreases the probability of reconstitution, subsequent to recombinations, of a functional viral genome capable of producing RCRs.

Although the positive control for searching for RCRs in preparations of amphotropic MLV vectors has been available since 1985 (6), no positive control similar to the RCRs which can be generated in preparations of MLV GaLV vectors is currently available.

The problem which the invention proposes to solve is therefore to develop a positive control for searching for RCRs in preparations of MLV GaLV vectors.

### SUMMARY OF THE INVENTION

To do this, the invention provides, first of all, a plasmid comprising a replicative retroviral genome.

This plasmid is characterized in that it contains:

a ψ sequence,

gag and pol sequences originating from the genome of an MLV virus,

a chimeric env sequence comprising a region corresponding to part of the envelope originating from the genome of an MLV virus and a region corresponding to part of the envelope originating from the genome of a GaLV virus.

### DETAILED DESCRIPTION OF THE INVENTION

In the remainder of the description and in the claims, the expression “replicative retroviral genome” denotes a genome composed of all the elements required for the production of replication-competent viral particles, in particular a psi sequence, at least one LTR sequence, the gag, pol and env genes and, more generally, all the genes present in the wild-type MLV virus.

In other words, the invention consists in having constructed a single plasmid carrying retroviral genomes of distinct origins, in other words, from different species. The plasmid is constructed by juxtaposing three retroviral sequences, respectively a first region comprising the gag and pol genes of an MLV virus, a second region comprising part of the envelope of an MLV virus, and a third region comprising part of the envelope of a GaLV virus.

The document by COSSET, WO 00/71578, describes the construction of retroviral vectors containing an envelope originating from an MLV virus, in which the receptor-binding domain (RBD) is replaced with a specific antibody. The chimeric nature of the envelope does not therefore come from the combination of two viral envelopes from different species, but from the substitution of a portion of given viral envelope (MLV) with a synthetic molecule, in the case in point, as specific antibody.

The document by CHRISTODOULOPOULOS (11) describes a method for preparing retroviral vectors in which the genome consists of the MLV gag and pol genes and a chimeric envelope resulting from the combination of part of an envelope originating from MLV and part of an envelope originating from GaLV.

The document by LANDAU (12) describes a method for producing MLV viruses possessing an MLV or RSV envelope. In other words, the envelope is not a chimera resulting from the combination of two parts of viral envelopes from different species.

The methods described in the three documents above cannot lead to the production of replicative viral particles. Specifically, none of the two gag-pol or env plasmids contains a ψ sequence essential for packaging the genome. Consequently, the distinct gag-pol and env plasmids are only able to produce the proteins encoded by the gag, pol and env genes in the transfected cells. The particles will be able to infect the target cells, but since the latter do not comprise the gag, pol and env genes, will not, in turn, be able to produce viral particles.

Moreover, since the gag-pol and env genes are carried by different plasmids, this implies that, in order to be replicative, the virions take away at least two distinct genomes (corresponding to the two plasmids), which is an improbable phenomenon.

The difficulty of the invention was therefore to prepare a single plasmid carrying a complete replicative retroviral genome, starting from a whole genome in which a gene derived from another species introducing a similar function is substituted.

According to the invention, the MLV envelope used can exhibit various tropisms, in particular xenotropic, ecotropic, polytropic, 10A1, advantageously amphotropic.

According to a first characteristic of the invention, the gag sequence encodes the gag polyprotein corresponding to the amino acid sequence SEQ ID NO. 1, or a sequence exhibiting at least 70%, advantageously at least 80%, homology with the sequence SEQ ID NO. 1.

In the remainder of the description and in the claims, the expression “sequence exhibiting a certain percentage homology with a given sequence” denotes an amino acid or nucleotide sequence which is identical to the given sequence to the degree of said percentage. The identity or homology is generally determined using a sequence analysis program, for example Pairwise BLAST, NCBI.

The percentage homology with the sequence SEQ ID NO. 1 was sought by comparing the amino acids encoded by the gag sequence of the plasmid pAMS with those of the gag sequence of various strains of virus of the MLV type. The results appear in the table below. The references given are those from GENBANK.

| Homologies (gag amino acids) | pAMS → gag (ref AAB64159) |
|---|---|
| AKV (ref J01998), A.A. gag | 73% |
| (ref AAB 03090) | |
| SL3-3 (ref AF169256), A.A. gag | 73% |
| (ref AAD55050) | |
| Friend (ref M93134), A.A. gag | 74% |
| (ref CAA46476) | |
| Friend FB29 (ref Z11128), A.A. gag | 73% |
| (ref CAA77478) | |
| Moloney (ref AF033811), A.A. gag | 81% |
| (ref AAC82566) | |
| MCF 1233 (ref U13766, A.A. gag | 72% |
| (ref AAA92678) | |

Similarly, and according to another characteristic of the invention, the pol sequence encodes the viral enzymes corresponding to the amino acid SEQ ID NO. 2, or a sequence exhibiting at least 80%, preferably at least 85%, advantageously at least 90%, homology with the sequence SEQ ID NO. 2.

The percentage homology with the sequence SEQ ID NO. 2 was sought by comparing the amino acids encoded by the pol sequence of the plasmid pAMS with those of the pol sequence of various strains of virus of the MLV type. The results appear in the table below. The references given are those from GENBANK.

| Homologies (pol amino acids) | pAMS → gag (ref AAB64160) |
|---|---|
| AKV (ref J01998), A.A. pol | 87% |
| (ref AAB 03091) | |
| SL3-3 (ref AF169256), A.A. pol | 87% |
| (ref AAD55051) | |
| Friend (ref M93134), A.A. pol | 93% |
| (ref CAA46477) | |
| Friend FB29 (ref Z11128), A.A. pol | 93% |
| (ref CAA77477) | |
| Moloney (ref AF033811), A.A. pol | 94% |
| (ref AAC82568) | |
| MCF 1233 (ref U13766, A.A. pol | 87% |
| (ref AAA92679) | |

As already mentioned, the gag and pol sequences originate from the genome of a MLV virus. In practice, the Moloney strain is used, although other strains can be used due to the strong homology of the gag and pol genes between the various known strains, as demonstrated above.

According to another characteristic of the plasmid of the invention, the env sequence is a chimeric sequence originating partly from the envelope of an MLV virus and partly from the envelope of a GaLV virus. In practice, the part of the envelope of the GaLV virus, cloned into the plasmid, comprises at least the region whose function is to define the specificity of the infection or the tropism of the viral envelope. In particular, the part of the envelope of the GaLV virus encodes the part of the env protein located between amino acids No. 32 and No. 644 (hereinafter referred to as “ID3-GaLV domain”) of the sequence SEQ ID NO. 3, i.e., overlapping the SU and TM subunits of the envelope protein, or a sequence exhibiting at least 70%, preferably at least 75%, advantageously at least 80%, homology with the ID3GaLV domain.

The percentage homology with the ID3-GaLV domain was sought by comparing the amino acids encoded by the env sequence of a GaLV virus, strain SEATO, with those of the env sequence and various strains of virus of the GaLV type. The results appear in the table below. The references given are those from GENBANK.

| | Env GaLV SEATO → env (ref |
|---|---|
| Homologies (env amino acids) | AAC96083) |
| Strain GaLV SF (ref AF055063), A.A. | 76% |
| (ref ACC 96086) | |
| Strain GaLV Brain (ref AF055062), | 83% |
| A.A. (ref AAC 96085) | |
| Strain GaLV Hall's Island | 84% |
| (ref AF055061), A.A. (ref AAC 96084) | |
| Strain GaLV X (ref U60065), A.A. | 76% |
| (ref AAC 80265) | |

In an advantageous example of preparation of the plasmid of the invention, the GaLV envelope fragment originates from the SEATO strain (GENBANK, ref M26927).

According to another characteristic of the plasmid of the invention, the chimeric env sequence comprises a region corresponding to part of the envelope of an MLV virus exhibiting a tropism chosen from amphotropic, xenotropic, ecotropic, polytropic and 10A1. In an advantageous embodiment, the MLV virus exhibits an amphotropic tropism. In practice, the portion of envelope of the amphotropic virus cloned into the plasmid is that which is required to enable, in combination with the region of the GaLV envelope which is substituted, the production of infectious viral particles and therefore the conformation of a functional viral envelope. The most specific region of the amphotropic envelope would therefore be required should substitution of the whole GaLV envelope give rise to the production of replicative viral particles, which is not the case according to the complementation experiments carried out by the applicant.

In an advantageous embodiment, the part of the envelope of the amphotropic MLV virus encodes the regions of the Env polyprotein which are located, firstly, between amino acids Nos 1 and 31, “ID 3-ampho-1 domain”, and, secondly, between amino acids Nos 645 and 676, “ID 3-ampho-2 domain”, of the sequence SEQ ID NO. 3, i.e. respectively at the beginning of the SU subunit, and at the end of the TM subunit of the envelope protein, or a sequence exhibiting at least 70%, preferably at least 80%, advantageously at least 85%, homology with the ID 3-ampho-1 and ID 3-ampho-2 domains.

The percentage homology with the ID 3-ampho-1 and ID 3-ampho-2 domains was sought by comparing the amino acids encoded by the env sequence of an amphotropic MLV virus, strain 4070A, with those of the env sequence of various strains of virus of the amphotropic MLV type. The results appear in the table below. The references given are those from GENBANK.

| | Env amphotropic 4070A → env |
|---|---|
| Homologies (env amino acids) | (ref AAA 46515) |
| Strain Moloney ampho MCF | 72% |
| (ref U 36991), A.A. (ref AAC 54626) | |
| Strain Moloney ampho Delta | 86% |
| (ref U 36800), A.A. (ref AAB 60590) | |
| Strain Moloney ampho RCR | 87% |
| (ref U 36602), A.A. (ref AAC 54625) | |
| Strain Moloney 10A1 (ref M 33470), | 81% |
| A.A. (ref AAA 46514) | |

In an advantageous example of preparation of the plasmid of the invention, the amphotropic envelope fragment originates from the 4070 A strain.

The invention also relates to a chimeric env sequence encoding the env protein corresponding to the amino acid sequence SEQ ID NO. 3 or a sequence exhibiting at least 95%, advantageously 98%, homology with the sequence SEQ ID NO. 3. This chimeric env sequence contains a part of the envelope of a GaLV virus encoding the part of the env protein located between amino acids No. 32 and No. 644 of SEQ ID NO. 3 and part of the envelope of an amphotropic MLV virus encoding the regions of the env polyprotein located, firstly, between amino acids No. 1 and 31 of the sequence SEQ ID NO. 3 and, secondly, between amino acids No. 645 and 676 of the sequence SEQ ID NO. 3.

As emerges from the above, the applicant noted that, in order to construct an RCR plasmid of the MLV GaLV type, simply substituting the env gene of an amphotropic MLV plasmid with the env gene of a GaLV plasmid did not give rise to the production of replicative viral particles (complementation experiments made it possible to show that this deficiency originated from the lack of functionality of the viral envelope). In view of these observations, not only was it not evident to propose a chimeric sequence combining advantageously an amphotropic envelope with a GaLV envelope, but in addition, it was evident to select the amphotropic part and the GaLV part required to produce a plasmid encoding a replicative virus having the same specificity of the infection as the GaLV envelope.

In a preferred embodiment, the invention relates to a plasmid comprising the viral genome corresponding to the nucleotide sequence SEQ ID NO. 4 or a sequence exhibiting at least 80%, preferably 85%, advantageously 90%, homology with the sequence SEQ ID NO. 4.

In a particular embodiment, such a plasmid is obtained from the plasmid pAM comprising the gag and pol genes and also an amphotropic envelope, and from part of a plasmid comprising the GaLV envelope, and corresponds to the nucleotide sequence SEQ ID NO. 5.

Of course, and in general, the plasmids covered by the invention can be obtained by all the usual molecular biology techniques, such as enzyme digestions, PCR (Polymerase Chain Reaction), ligations, amplifications, cloning, etc.

The invention also relates to a bacterium producing the chimeric plasmid of the invention, in particular a plasmid comprising the viral genome corresponding to the nucleotide sequence SEQ ID NO. 4 described above, more particularly the plasmid corresponding to the nucleotide sequence SEQ ID NO. 5. An advantageous bacteria corresponds to E. coli DH10B.

The viral genome contained in the chimeric plasmid of the invention can be expressed in any suitable cell line, such as, for example, and in a nonlimiting manner, human, monkey, rat, hamster or chicken cells, and particularly fibroblast lines.

The invention also relates to a virion produced by one of the cell lines described above.

More particularly, the invention relates to a virion containing the viral genome corresponding to the nucleotide sequence SEQ ID NO. 4 or a sequence exhibiting at least 60%, preferably at least 70%, advantageously at least 80%, or even 85% or 90%, homology with the sequence SEQ ID NO. 4.

The retrovirus thus produced finds a particular application as a positive control in any test intended to detect RCRs or other types of nonreplicative recombinants in preparations of retroviral vectors of the MLV GaLV type.

As already mentioned, these tests, which are required by the FDA, are aimed at testing not only the supernatant containing the retroviral vector produced by the cell line, but also the cell line itself and the patients treated with the gene therapy vectors under consideration. In the first two cases, the detection of the RCRs should be preceded by a step consisting of amplifying the possible RCRs present in the sample tested. If the sample under consideration is a supernatant, amplification thereof is carried out by bringing it into contact with a GaLV envelope-permissive cell line. If the sample under consideration is the vector-producing line, amplification thereof is carried out by coculturing it with the permissive line. The FDA requires that a test be carried out in parallel for using a positive control; in the case in point, the virus produced by the cell line expressing the plasmid which is the subject of the invention. Various types of method which apply this protocol are known under the names XC (7), PG4 S⁺ L⁻(8), PCR or else mobilization test. The mobilization test is the test which is preferred among the abovementioned tests.

Thus, the invention relates to a mobilization test intended to detect RCRs in preparations of retroviral vectors of the MLV GaLV type, and which consists:

first of all, in infecting or coculturing a GaLV envelope-permissive cell line with, respectively, the retroviral vector or the producer line to be tested, said permissive line containing a mobilization vector itself comprising a gene for resistance to a given antibiotic, then

in recovering the supernatant from the culture or coculture in order to transfer it onto indicator cells, also GaLV-envelope permissive, and treated with said antibiotic,

in searching for the possible resistance of the indicator cells to the antibiotic, the resistance to the antibiotic revealing the presence of RCRs in the sample tested,

in carrying out in parallel the same test with the positive control corresponding to the virion of the invention.

In fact, each sample should be tested, firstly, alone and, secondly, with the positive control added in order to verify that the sample does not exert an inhibitory effect on the RCR detection. In the remainder of the description and in the claims, the expression “mobilization vector” denotes a vector comprising in particular the LTRs and the Ψ sequence of MLV and also a gene for resistance to an antibiotic.

In practice, the mobilization tests are carried out on permissive cells, human fibroblasts for example (HT1080 or HCT116 in particular), containing the mobilization vector introducing resistance to hygromycin B.

The invention also relates to a kit for carrying out the mobilization test, which contains:

the virion of the invention as described above;

a GaLV envelope-permissive cell line, in particular the abovementioned cells; and

the required reagents.

FIG. 1 represents the restriction map of plasmid pAMS

FIG. 2 represents the restriction map of plasmid phCMV GaLV

FIG. 3 represents the restriction map of the plasmid pRCR-GaLV-1

FIG. 4 represents the restriction map of the plasmid pRCR-GaLV-2 (plasmid of the invention).

A/Construction of the Plasmid of the Invention

This example reflects a possible embodiment of the construction of the plasmid of the invention (pRCR-GaLV-2), the sequence of which corresponds to the sequence SEQ ID NO. 5.

I/Step 1 of the Construction:

For the most part, the first step consists in constructing a first plasmid, called pRCR-GaLV-1, resulting from the ligation of 3 fragments, respectively:

a first Cla I-Sal I fragment of 7392 pb, located between nucleotides 8232 and 4296 of pAMS (FIG. 1) and therefore containing the gag genes and part of the pol gene of an MLV virus, strain Moloney (paragraph I-1 below);

a second, blunt end—Cla I fragment of 1810 pb, located between nucleotides 2499 and 4309 of the plasmid phCMV-GaLV (FIG. 2) and containing part of the env gene of a GaLV virus, strain SEATO (paragraph 1-2 below); and

a third, blunt end—Sal I fragment of 2162 pb, located between nucleotides 4296 and 6457 of pAMS and containing the missing part of the pol gene of the MLV virus, strain Moloney, and part of the envelope of an amphotropic MLV virus, strain 4070A (paragraph I-3 below).

I-1—Production of the 7392 pb Cla I-Sal I Fragment from the Plasmid pAMS

Digestion of the plasmid pAMS (FIG. 1) with the Cla I and Sal I enzymes generates 3 fragments of 1275, 2661 and 7392 pb. This digestion is carried out in 2 steps: a first digestion of 15 μg of plasmid in 100 units of the Sal I enzyme followed by precipitation of the digested DNA, and a second digestion with 80 units of the Cla I enzyme. The 7392 pb fragment is recovered in the following way: after migration of the double enzyme digestion product in a 0.8% agarose gel, the piece of gel containing the 7392 pb fragment is cut out with a scalpel and its DNA is then extracted by filtration at 0.2 μm. For this, a filter (0.2 μm Acrodisc, Ref. 4192, Gelman Sciences) is placed at the end of a 5 ml syringe before being wetted with 200 μl of 0.1×TE. The piece of agarose is then passed through the filter and the filter is then rinsed with 400 μl of 0.1×TE. The DNA collected is precipitated with isopropanol and rinsed with 70% ethanol.

I-2—Production of the 1810 pb Blunt End—Cla I Fragment from the Plasmid phCMV-GaLV

A PCR is carried out on the plasmid phCMV-GaLV (FIG. 2), the sequence of which corresponds to the sequence SEQ ID NO. 6, using oligonucleotides 1 (SEQ ID NO. 7) and 2 (SEQ ID NO. 8).

Oligo 1 (reverse)

SEQ ID NO. 7: GGTCAACTTGGCCATGGTGGC (21 mer)

4501→4481 phCMV-GaLV

Oligo 2 (sense)

SEQ ID NO. 8: CAGCCCATGACCCTCACTTGG (21 mer)

2499→2519 phCMV-GaLV

The amplification is carried out with 40 ng of plasmid, 1.25 units of pfu Turbo polymerase (Stratagene, Ref. 600250), 0.2 mM of dNTP, 0.5 μM of each oligonucleotide in a final volume of 50 μl. The amplification conditions are as follows: 5 min at 91 C+30* (1 min at 91 C+45 sec at 71.6 C+2 min at 72° C.)+10 min at 72° C. The PCR is carried out with the Mastercycler gradient (Eppendorf). After verification of the size (2002 pb) of the amplified fragment by migrating an aliquot on a 0.8% agarose gel, the PCR product is digested with 100 units of the Cla I enzyme. This digestion is loaded onto a 0.8% agarose gel and the piece of gel containing the digested PCR fragment (1810 pb) is recovered. The DNA is extracted from the agarose gel according to the method described at the end of paragraph I-1.

I-3—Production of the 2162 pb Blunt End—Sal I Fragment from the Plasmid pAMS

A PCR is carried out on the Xho I-Xho I fragment (4773 pb) of the plasmid pAMS using oligonucleotides 3 (SEQ ID NO. 9) and 4 (SEQ ID NO. 10).

Oligo 3 (reverse)

SEQ ID NO. 9: CCCTACTCCTAACAGGACTCC (21 mer)

6457→6437 pAMS

Oligo 4 (sense)

SEQ ID NO. 10: GTCAGAGATGGCTGACTGAGG (21 mer)

4015→4035 pAMS

The amplification is carried out with 20 ng of the digested plasmid, 1.25 units of pfu Turbo polymerase (Stratagene, Ref. 600250), 0.2 mM of dNTP, and 0.5 μM of each oligonucleotide in a final volume of 50 μl. The amplification conditions are as follows: 5 min at 91° C.+30* (1 min at 91° C+45 sec at 60.1° C.+2 min at 72° C.)+10 min at 72° C. The PCR is carried out with the Mastercycler gradient (Eppendorf). After verification of the size (2442 pb) of the amplified fragment by migrating an aliquot on a 0.8% agarose gel, the PCR product is digested with 80 units of the Sal I enzyme. This digestion is loaded onto a 0.8% agarose gel and the piece of gel containing the digested PCR fragment (2162 pb) is recovered. The DNA is extracted from the agarose gel according to the method described at the end of paragraph I-1.

I-4—Ligation of the 3 Fragments (7392 pb Cla I-Sal I+1810 pb Blunt End—Cla I+2162 pb Blunt End—Sal I) and Production of the Plasmid pRCR-GaLV-1

The 3 fragments to be assembled are quantified using the Bio Rad software (Bio Rad, Quantity one SW, MAC, Ref. 1708609) and the ligation is performed with a ⅙ proportion of the 7392 pb fragment (which contains the plasmid vector) and an equivalent proportion of each of the other 2 fragments. The ligation is carried out with 40 units of T4 DNA ligase (Biolabs, Ref. 202S); it is used to transform E. coli DH10B bacteria (Life Technology, Ref. 182979-010), which are plated out onto a dish of LB supplemented with ampicillin (the plasmid vector contained in the 7392 pb fragment in fact contains an ampicillin resistance gene). After overnight incubation at 37° C., the dish exhibits 7 colonies which are used to produce the corresponding 7 minipreps. These minipreps are analyzed by enzyme restriction. A single clone exhibits the expected profile, it is called pRCR-GaLV-1 (FIG. 3) and corresponds to the sequence SEQ ID NO. 11.

The following are in pRCR-GaLV-1: the gag gene of pAMS originating from the MLV virus, located between nucleotides 1212 and 2828, the pol gene of pAMS originating from the MLV virus, located between nucleotides 2829 and 6428, a first part of the amphotropic envelope of pAMS originating from the MLV virus, located between nucleotides 6368 and 6457, part of the envelope of GaLV, located between nucleotides 6458 and 8269, and a second part of the amphotropic envelope of pAMS, located between nucleotides 8270 and 8332.

II/Step 2 of the Construction

The tests carried out with the plasmid pRCR-GaLV-1 showed that it corresponds to a viral genome which produces functional Gag and Pol proteins and a nonfunctional viral envelope. A small additional region of the GaLV envelope is added to pRCR-GaLV-1 in order to construct the pRCR-GaLV-2.

II-1—Production of the 1435 pb Nco I-Nco I fragment from the plasmid PRCR-GaLV-1

A PCR is carried out on the plasmid pRCR-GaLV-1 using oligonucleotides 5 (SEQ ID NO. 12), 6 (SEQ ID NO. 13), 7 (SEQ ID NO. 14) and 8 (SEQ ID NO. 15).

Oligo 5 (reverse)

SEQ ID NO.12

GGGTCATGGGCTGGTGGGGGTTCTTATTTTGCAGACTCGTCATCC
CTACTCCTAACAGGACTCC (64 mer):

6500→6437 pRCR-GaLV-2

(the sequence introduced with this oligonucleotide is underlined).

Oligo 6 (sense)

SEQ ID NO.13

GTTAGGAGTAGGGATGACGAGTCTGCAAAAGAACCCCCACC
AGCCCATGACCCTCACTTGG (64 mer)

6445→6508 pRCR-GaLV-2

(the sequence introduced with this oligonucleotide is underlined).

Oligo 7 (sense)

SEQ ID NO. 14

CAACTGGCTCTAGAGACTGG (20 mer)

5908→5927 pRCR-GaLV-2

Oligo 8 (reverse)

SEQ ID NO. 15

CCTTTCCTATGCACAACCCG (20 mer)

7553→7534 pRCR-GaLV-2

The amplification is carried out with 40 ng of the plasmid, 1 unit of DyNazyme polymerase (Ozyme, Ref. F505L), 0.2 mM of dNTP, 0.5 μM of each oligonucleotide, and 4% of DMSO in a final volume of 50 μl. The amplification conditions are as follows: 5 min at 91° C.+35* (1 min at 91° C.+45 sec at 60.7° C.+1 min 30 sec at 72° C.)+10 min at 72° C. The PCR is carried out with the Mastercycler gradient (Eppendorf). An aliquot of the PCR product is loaded onto a 1.5% agarose gel, and several bands appear having approximately the following sizes: 1.6, 1.1 and 0.6 kb. The remainder of the PCR product is digested for 2 hours at 37° C. with 20 units of Dpn I (Ozyme, Ref. R0176L) in order to eliminate possible traces of matrix. The product of this digestion is loaded onto a 1.0% agarose gel and, after migration, the 1.1 and 0.6 kb bands are cut out and extracted according to the method described at the end of paragraph I-1. These 2 fragments correspond respectively to the amplifications carried out with oligonucleotides 6 and 8 (theoretical size of the amplification 1108 pb) and with oligonucleotides 5 and 7 (theoretical size of the amplification 592 pb). We prefer these 2 fragments to that of 1.6 kb because they have to contain the 30 nucleotides which must be integrated with oligonucleotides 5 and 6, whereas the 1.6 kb fragment might have been generated by only oligonucleotides 7 and 8 and might therefore not contain these 30 nucleotides. A further PCR with only the external primers (oligonucleotides 7 and 8) is carried out on the 1.1 and 0.6 kb fragments extracted from the agarose gel.

The amplification is carried out with 50 ng of the 0.6 kb fragment and 50 ng of the 1.1 kb fragment, 1 unit of DyNazyme polymerase (Ozyme, Ref. F505L), 0.2 mM of dNTP, 0.5 μM of each oligonucleotide, and 4% of DMSO in a final volume of 50 μl. The amplification conditions are as follows: 4 min at 94° C.+35* (1 min at 94° C.+45 sec at 57.8° C.+1 min 30 sec at 72° C.)+10 min at 72° C. The PCR was carried out with the Mastercycler gradient (Eppendorf). An aliquot of the PCR product is loaded onto a 1.5% agarose gel, and the size of the amplified fragment is correct, approximately 1.6 kb (theoretical size of 1645 pb).

A fraction (20 μl) of the PCR product is then digested with 40 units of the Nco I enzyme. This digestion is loaded onto a 0.8% agarose gel and the piece of gel containing the digestion PCR fragment (1435 pb) is recovered. The DNA is extracted from the agarose gel according to the method described at the end of paragraph I-1.

II-2—Production of the 9959 pb Nco I-Nco I Fragment from the Plasmid pRCR-GaLV-1

Digestion of the plasmid pRCR-GaLV-1 with the Nco I enzyme generates 2 fragments of 1405 and 9959 pb. This digestion is carried out with 10 μg of plasmid, with 40 units of the Nco I enzyme. After migration of the enzyme digestion product in a 0.8% agarose gel, the piece of gel containing the 9959 pb fragment is cut out with a scalpel and its DNA is then extracted according to the method described at the end of paragraph I-1.

II-3—Ligation of the 2 Fragments (1435 pb Nco I-Nco I+9959 pb Nco 1-Nco I) and Production of the Plasmid pRCR-GaLV-2 (FIG. 4)

The 2 fragments to be assembled are quantified with the PicoGreen kit (Molecular Probes, Ref. P-7589). In order to avoid the 9959 pb fragment (which contains the ampicillin resistance gene) ligating on itself, it is dephosphorylated before ligation. The dephosphorylation is carried out in the following way: incubation of the 9959 pb DNA fragment for 1 hour at 37° C. with 1 unit of SAP (Shimp alkaline phosphotase, Amersham Life Science, Ref. 70103) per 5 pmol. The SAP is then inactivated by incubation for 15 minutes at 65° C. The ligation is carried out with a ⅙ proportion of the 9959 pb fragment (which contains the plasmid vector) and a ⅚ proportion of the 1435 pb fragment. It is carried out with 40 units of T4 DNA ligase (Biolabs, Ref. 202S). The ligation product is used to transform E. coli DH10B bacteria (Life Technology, Ref. 182979-010), which are plated out on a dish of LB supplemented with ampicillin (the plasmid vector contained in the 9959 pb fragment in fact contains an ampicillin resistance gene). After overnight incubation at 37° C., the dish exhibits several tens of colonies which are analyzed by a PCR. Out of the 80 colonies analyzed, 56 carry the insert. Three positive colonies are selected to continue the experiments, the corresponding plasmids are called: pRCR-GaLV-2-C1, pRCR-GaLV-2-D1 and pRCR-GaLV-2-H1.

B/Production of the RCR-GaLV-2 Viral Supernatant

I—Cell Transfection

The 293 and HT1080 human cell lines are transfected with the plasmids pRCR-GaLV-2-C1, pRCR-GaLV-2-D1 and pRCR-GaLV-2-H1. These cells are cultured in DMEM (Gibco BRL, Ref. 31966-021) containing 10% of fetal calf serum (Hyclone, Ref. SH 30071.03) and 1% of penicillin/streptomycin (Gibco BRL, Ref. 15070-063). The cells are seeded the day before transfection in 6-well plates in a proportion of 6×10⁵ cells/well for the 293 cells and of 3×10⁵ cells/well for the HT1080 cells. The transfections are carried out with calcium phosphate, with 4.2 μg of plasmid/well. Subsequently, HCT116 human cells were also used to produce the viral supernatant corresponding to the plasmid pRCR-GaLV-2.

2—Supernatent Collection

The transfected cells are maintained in culture and reverse transcriptase (enzyme produced by retroviruses; detecting of this enzyme makes it possible to demonstrate the presence of retroviruses) is sought in their supernatant 3 weeks after transfection. The day before sampling of the supernatant, the culture medium of the transfected cells change. The supernatant intended for measurement of reverse transcriptase is filtered at 0.45 μm (Sartorius, Ref. 16555) and stored at −20° C.

C/Characterization of the RCR-GaLV-2 Viral Supernatant

1—Measurement of the Reverse Transcriptase Activity in the Transfected Cells Supernatant

The reverse transcriptase activity is measured with the following mix: 50 mM Tris, pH 7.8; 7.5 mM KCl; 5 μg/ml polyA; 1.57 mg/ml oligodT; 0.05% NP40. Just before this mix is used, 5 mM MnCl₂ and 1 ml of dTTP³²/ml of mix are added thereto. This final mix is distributed into the wells of a 96-well plate in a proportion of 25 μl/well. 5 μl of each of the supernatants to be tested are added to each of the wells. The plate is then incubated for 1 hour at 37° C. After this incubation, 7 μl of each of the wells are deposited, in the form of spots, onto DE81 paper, and this paper is then dried in an incubator. The depositing of 7 μl of the content of each well (in the same place as the preceding deposit) followed by drying of the paper is repeated twice so as to deposit 21 μl of the content of each well. The DE81 paper is then washed twice for 5 minutes in 2×SSC at ambient temperature, and then once for 1 to 2 minutes with absolute ethanol. The paper is dried and exposed in a cassette overnight. The following day, developing of the x-ray reveals that the supernatant from the 293 cells transfected with the plasmids pRCR-GaLV-2-C1 or pRCR-GaLV-2-H1 contains reverse transcriptase.

D/Evaluation of the Tropism of the RCR-GaLV-2 Supernatant and of its Ability to Serve as a Positive Control in Searching for RCRs in Preparations of MLV Vectors Pseudotyped with the GaLV Envelope

All the cells used in the mobilization test are cultured in DMEM (Gibco BRL, Ref. 31966-021) containing 10% of fetal calf serum (Hyclone, Ref. SH 30071.03) and 1% of penicillin/streptomycin (Gibco BRL, Ref. 15070-063).

The mobilizing line HT1080-pLHL was formed by infection of HT1080 cells with the supernatant from an amphotropic packaging line transfected with the mobilization vector pLHL (10), then selection of the cells with hygromycin B (Gibco BRL, Ref. 10687-010), and then cloning.

The HT1080-pLHL cells are seeded in 12-well plates in a proportion of 6×10⁴ cells/well. The following day, these cells are infected, in the presence of 8 μg/ml of hexadimethrine bromide (Sigma, Ref. H-9268), with serial dilutions of the RCR-amphotrope positive control (ATCC, Ref. VR-1450) or with serial dilutions of the supernatant formed subsequently to the transfection of 293 cells with the plasmid pRCR-GaLV-2-C1 (see above). The dilution range for the RCR-amphotrope control goes from 2×10⁴ RCRs/well to 2 RCRs/well. The same dilutions are prepared for the RCR-GaLV control, which will make it possible to compare its titer with that of the RCR-amphotrope control. The day following infection, the supernatant from each well is removed and replaced with new culture medium. The same day, human indicator cells, HCT116, and murine indicator cells, Mus dunni, are seeded in 12well plates in a proportion, respectively, of 5×10⁴ and 5×10³ cells/well. Four days after infection of the HT1080-pLHL cells, the supernatant from the cells is removed, filtered through 0.45 μm, and used to infect the indicator cells in the presence of 8 μg/ml of hexadimethrine bromide (Sigma, Ref. H-9268). The day after this infection, the supernatant from each well of HCT 116 and Mus dunni cells is removed and replaced with new culture medium supplemented in 0.3 mg/ml of Hygromycin B (Gibco BRL, Ref. 10687-010). This change of culture medium is repeated twice a week for 3 weeks. At the end of the 3 weeks of selection, the test is ended and the cells resistant to hygromcyin B are identified.

On the HCT 116 cells, which can be infected both with the amphotropic envelope and with the GaLV envelope, the final dilution of the RCR-GaLV-2-C1 positive control which gives hygromycin B-resistant cells is the same as the final dilution of the RCR-amphotrope positive control which gives hygromycin B-resistant cells. For the RCR-amphotrope control, the precise titer of which is known, this dilution corresponds to 1 RCR. According to this observation, the titer of the RCR-GaLV-2-C1 control would be comparable to that of the RCR-amphotrope control (titer of the latter control 3.7×10⁶ infectious particles per ml). This observation correlates with the titer measured by TAQMAN quantitative RCR, which reveals that the RCR-GaLV2 positive control comprises approximately 2×10^{E}6 infectious particles per ml.

On the Mus dunni cells, which can be infected with amphotropic envelope but not with the GaLV envelope, a single positive colony is observed for the lowest dilution of the RCR-GaLV-2-C1 positive control, whereas, for the RCR-amphotrope control, a 1000-fold greater dilution (corresponding to 20 RCRs) gives hygromycin B-resistant cells.

According to this immobilization test, it may be concluded that:

the RCR-GaLV-2-C1 control is capable of mobilizing the pLHL vector (since hygromycin B-resistant colonies are obtained on the HCT116 indicator cells).

The RCR-GaLV-2-C1 control would have no problem of replication, since its titer is comparable to that of the RCR-amphotrope control produced by the ATCC (according to comparison of the final dilution of each of the controls which gives a positive result when the immobilization test is revealed on HCTI 16 indicator cells).

The RCR-GaLV-2-C1 control would exhibit the same tropism (specificity of infection) as the GaLV envelope; specifically, this envelope enables infection of human cells (for example HCT116) but not, or much less, infection of a mouse cell (for example Mus dunni).

### Bibliography

(1) Donahue R. E. et al. Helper virus induced T cell lymphoma in non human primates after retroviral mediated gene transfer. J Exp Med 1992; 176: 1125-1135

(2) Cone, R. R. & Mulligan, R. C., 1984, PNAS 81: 6349-6353

(3) Bosselman, R. A. et al., 1987, Mol. Cell. Biol. 7: 1797-1806

(4) Markowitz D., Goss S., Bank A.; Construction and use of a safe and efficient amphotropic packaging cell line. Virology 1988; 167: 400-406

(5) Supplemental guidance on testing for replication competent retrovirus in retroviral vector based gene therapy products and during follow-up of patients in clinical trials using retroviral vectors, available from US Department of Health and Human Services, Food and Drug Administration, Center for Biologics Evaluation and Research (CBER), October 2000

(6) Miller A. D., Law M-F., Verman I. Generation of helper-free amphotropic retroviruses that transduce a dominant-acting, methotrexate-resistant dihydrofolate reductase gene. Mol Cell Biol 1985; 5: 431-437

(7) Eglisis, M. A. et al., 1995, Gene Ther. 2: 486-492

(8) Rowe W. P., Pugh W. E., Hartley J. W. Plaque assay techniques for murine leukaemia viruses. Virology 1970; 42: 1136-1139

(9) Haapala D. K., Robey W. G., Oroszlan S. D., Tsai W. P. Isolation from cats of endogenous type C virus with a novel envelope glycoprotein. J Virol 1985; 53: 827-833

(10) Palmer T. D., Hock R. A., Osborne W. R., Miller A. D. 1987. Efficient retrovirus-mediated transfer and expression of a human adenosine deaminase gene in diploid skin fibroblasts from an adenosine deaminase-deficient human. Proc Natl Acad Sci USA 84(4): 1055-1059

(11) Ilias CHRISTODOULOPOULOS et Paula M. CANNON, “Sequences in the cytoplasmic tail of the Gibbon Ape Leukemia Virus envelope protein that prevent its incorporation into Lentivirus Vectors”. Journal of Virology, May 2001, p. 4129-4138.

(12) LANDAU N. R. et al., “Packaging system for rapid production of murine leukaemia virus vectors with variable tropism”. Journal of Virology, The American Society for Microbiology, US. Vol. 66, No. 8, Août 1992.

1. A plasmid comprising a replicative retroviral genome, wherein said genome comprises:
(a) a psi (ψ) sequence;
(b) gag and pol sequences originating from the genome of an MLVvirus; and
(c) a chimeric env sequence comprising a region corresponding to part of the envelope originating from the genome of an MLV virus and a region corresponding to part of the envelope originating from the genome of a GaLV virus.

2. The plasmid as claimed in claim 1, characterized in that the envelope of the MLV virus exhibits a tropism which is either amphotropic, ecotropic, polytropic, 10A1 or xenotropic.

3. The plasmid of claim 1, characterized in that the gag sequence encodes the gag polyprotein corresponding to the amino acid sequence SEQ ID NO: 1, or a sequence exhibiting at least 70% homology with SEQ ID NO: 1.

4. The plasmid of claim 1, characterized in that the pol sequence encodes the viral enzymes corresponding to the amino acid sequence SEQ ID NO: 2 or a sequence exhibiting at least 80% homology with SEQ ID NO: 2.

5. The plasmid of claim 1, characterized in that MLV virus originates from the Moloney strain.

6. The plasmid of claim 1, characterized in that the part of the envelope of the GaLV virus comprises at least the region whose function is to define the tropism of the viral envelope.

7. The plasmid of claim 1, characterized in that the part of the envelope of the GaLV virus encodes the part of the env protein located between amino acids No. 32 and No. 644 (“ID3-GaLV domain”) of the sequence SEQ ID NO: 3 or a sequence exhibiting at least 70% homology with the ID3-GaLV domain.

8. The plasmid of claim 1, characterized in that the part A of the env is an envelope fragment of GaLV virus derived from the SEATO strain.

9. The plasmid of claim 1, characterized in that the envelope of the MLV virus exhibits an amphotropic tropism.

10. The plasmid of claim 9, characterized in that the part of the envelope of the amphotropic MLV virus is that which is required to enable, in combination with the region of the GaLV envelope which is substituted, the production of infectious viral particles.

11. The plasmid of claim 9, characterized in that the part of the envelope of the amphotropic MLV virus encodes the regions of the Env polyprotein which are located, firstly, between amino acids Nos. 1 and 31 “ID 3-ampho-1 domain” and, secondly, between amino acids Nos. 645 and 676 “ID 3 ampho-2 domain”) of the sequence SEQ ID NO: 3, or a sequence exhibiting at least 70% homology with the ID 3-ampho-1 and ID 3-ampho-2 domain.

12. The plasmid of claim 9, characterized in that the amphotropic envelope part originates from the 4070 A strain.

13. A plasmid comprising the viral genome corresponding to SEQ ID NO: 4 or a sequence exhibiting at least 80% homology with SEQ ID NO: 4.

14. A plasmid corresponding to SEQ ID NO: 5.

15. A bacterium producing the plasmid of claim 1.

16. The bacterium of claim 15, characterized in that it is E. coli DH 10B.

17. A cell line expressing the retroviral genome contained in the plasmid of claim 1.

18. The cell line of claim 17, characterized in that the cells are human cells.

19. The cell line of claim 18, characterized in that the cells are fibroblasts.

20. A virion produced by a cell line of claim 17.

21. A virion containing the viral genome corresponding to SEQ ID NO: 4 or a sequence exhibiting at least 60% homology with SEQ ID NO: 4.

22. A mobilization test intended to detect RCRs in preparations of retroviral vectors of the MLV GaLV type, and which consists:
(a) first of all, in infecting or coculturing a GaLV envelope-permissive cell line with, respectively, the retroviral vector or the producer line to be tested, said permissive line containing a mobilization vector itself comprising a gene for resistance to a given antibiotic, then
(b) in recovering the supernatant from the culture or coculture in order to transfer it onto indicator cells, also GaLV-envelope permissive, and treated with said antibiotic,
(c) in searching for the possible resistance of the indicator cells to the antibiotic, the resistance to the antibiotic revealing the presence of RCRs in the sample tested,
(d) in carrying out in parallel the same test with the positive control corresponding to the virion of claim 20.

23. A kit for carrying out a mobilization test of claim 23, comprising:
(a) the virion which is the subject of either of claims 20 and 21;
(b) GaLV envelope-permissive mobilizing cells; and
(c) the required reagents.

24. The kit of claim 24, characterized in that the permissive cells are HT1080 or HCT116 cells.

25. A chimeric env sequence encoding the env protein corresponding to the amino acid sequence SEQ ID NO: 3 or a sequence exhibiting at least 95% homology with SEQ ID NO: 3.


SEQUENCE LISTING (15 sequences)

SEQ ID NO: 1 - protein, 538 aa, Artificial Sequence
  product of gag sequence of pAM plasmid
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 2 - protein, 1199 aa, Artificial Sequence
  product of pol gene of pAM plasmid
GGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVMGPMGQPLQVLTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHDCLDILAEAHGTRSDLTDQPLPDADHTWYTDGSSFLQEGQRKAGAAVTTETEVIWARALPAGTSAQRAELIALTQALKMAEGKKLNVYTDSRYAFATAHIHGEIYRRRGLLTSEGKEIKNKDEILALLKALFLPKRLSIIHCPGHQKGNSAEARGNRMADQAAREVATRETPGTSTLLIENSTPYTHEHFHYTVTDTKDLTKLGATYDSAKKYWVYQGKPVMPDQFTFELLDFLHQLTHLSFSKTKALLERSPSPYYMLNRDRTLKNITETCKACAQVNASKSAVKQGTRVRGHRPGTHWEIDFTEVKPGLYGYKYLLVFVDTFSGWIEAFPTKKETAKVVTKKLLEEIFPRFGMPQVLGTDNGPAFVSKVSQTVADLLGIDWKLHCAYRPQSSGQVERMNRTIKETLTKLTLATGSRDWVLLLPLALYRARNTPGPHGLTPYEILYGAPPPLVNFPDPDMTRVTNSPSLQAHLQALYLVQHEVWRPLAAAYQEQLDRPVVPHPYRVGDTVWVRRHQTKNLEPRWKGPYTVLLTTPTALKVDGIAAWIHAAHVKAADTESGPSSGRTWRVQRSQNPLKIRLTRGSP

SEQ ID NO: 3 - protein, 676 aa, Artificial Sequence
  product of env gene of GaLV + ampho
MARSTLSKPPQDKINPWKPLIVMGVLLGVGMTSLQNKNPHQPMTLTWQVLSQTGDVVWDTKAVQPPWTWWPTLKPDVCALAASLESWDIPGTDVSSSKRVRPPDSDYTAAYKQITWGAIGCSYPRARTRMASSTFYVCPRDGRTLSEARRCGGLESLYCKEWDCETTGTGYWLSKSSKDLITVKWDQNSEWTQKFQQCHQTGWCNPLKIDFTDKGKLSKDWITGKTWGLRFYVSGHPGVQFTIRLKITNMPAVAVGPDLVLVEQGPPRTSLALPPPLPPREAPPPSLPDSNSTALATSAQTPTVRKTIVTLNTPPPTTGDRLFDLVQGAFLTLNATNPGATESCWLCLAMGPPYYEAIASSGEVAYSTDLDRCRWGTQGKLTLTEVSGHGLCIGKVPFTHQHLCNQTLSINSSGDHQYLLPSNHSWWACSTGLTPCLSTSVFNQTRDFCIQVQLIPRIYYYPEEVLLQAYDNSHPRTKREAVSLTLAVLLGLGITAGIGTGSTALIKGPIDLQQGLTSLQIAIDADLRALQDSVSKLEDSLTSLSEVVLQNRRGLDLLFLKEGGLCAALKEECCFYIDHSGAVRDSMKKLKEKLDKRQLERQKSQNWYEGWFNNSPWFTTLLSTIAGPLLLLLLLLILGPCIINRLVQFVKDRISVVQALVLTQQYHQLKPIEYEP

SEQ ID NO: 4 - DNA, 8889 bp, Artificial Sequence
  replicative viral genome
tttgaaagaccccacccgtaggtggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggcgtcctaacgcaaaagctgggaccttggcgtcggccggtggcctacctgtctaaaaagctagacccagtggcagctggctggcccccctgcctacggatggtggcagccattgcagttctgacaaaagatgctggcaagctcactatgggacagccgttggtcattctggccccccatgccgtagaggcactagttaagcaaccccctgatcgctggctctccaatgcccggatgacccattaccaagccctgctcctggacacggaccgggtccagttcgggccagtagtggccctaaatccagctacgctgctccctctgcctgaggaggggctgcaacatgactgccttgacatcttggctgaagcccacggaactagatcagatcttacggaccagcccctcccagacgccgaccacacctggtacacggatgggagcagcttcctgcaagaagggcagcgtaaggccggagcagcggtgaccactgagactgaggtaatctgggccagggcattgccagccgggacatcggcccaaagagctgaactgatagcgctcacccaagccctaaagatggcagaaggtaagaagctaaatgtttatactgatagccgttacgcttttgccaccgcccatattcatggagaaatatacagaaggcgcgggttgctcacatcagaaggaaaagagatcaagaacaaggacgagatcttagccctactaaaggctctcttcttgcccaaaagacttagcataattcattgcccgggacatcaaaaaggaaacagcgcagaggccaggggcaaccggatggccgaccaagcggcccgagaagtagccactagagaaactccaggaacttccacacttctgatagaaaactcaaccccctatacccatgaacactttcactatacagtaactgacacaaaggatttgaccaaactaggagccacttatgacagtgcgaagaaatattgggtctatcaaggaaagcctgttatgcctgatcaattcacctttgagttactagactttcttcaccaattgacccacctcagcttctcaaaaacaaaggctctcctagagagaagccccagtccctactacatgctgaaccgggatcgaacactcaaaaatatcactgagacctgcaaagcttgtgcacaagtcaatgccagcaagtctgccgttaagcaaggaactagggtccgcgggcatcggcctggcacacactgggagatcgatttcaccgaggtaaaacctggattgtatggctataagtatcttttagtttttgtagatactttttctggctggatagaagctttcccaactaagaaagaaaccgccaaggtcgtgaccaagaaactgctagaagagatcttccctaggttcggcatgccgcaggtattgggaactgacaatgggcctgccttcgtctccaaggtgagtcagacagtggccgatctgttggggattgattggaaattacattgtgcatacagaccccaaagctcaggtcaggtagaaagaatgaataggaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgaccagagttactaacagcccctctctccaagctcacttacaggctctctacttagtccagcacgaagtttggagaccactggcggcagcttaccaagaacaactggaccggccggtggtgcctcacccttaccgggtcggcgacacagtgtgggtccgccgacatcaaaccaagaacctagaacctcgctggaaaggaccttacacagtcctgctgaccacccccaccgccctcaaagtagacggtatcgcagcttggatacacgcagcccacgtaaaggcggccgacaccgagagtggaccatcctctggacggacatggcgcgttcaacgctctcaaaaccccctcaagataagattaacccgtggaagcccttaatagtcatgggagtcctgttaggagtagggatgacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 5 - DNA, 11394 bp, Artificial Sequence
  pRCR-GaLV-2
gaattcataccagatcaccgaaaactgtcctccaaatgtgtccccctcacactcccaaattcgcgggcttctgcctcttagaccactctaccctattccccacactcaccggagccaaagccgcggcccttccgtttctttgcttttgaaagaccccacccgtaggtggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggcgtcctaacgcaaaagctgggaccttggcgtcggccggtggcctacctgtctaaaaagctagacccagtggcagctggctggcccccctgcctacggatggtggcagccattgcagttctgacaaaagatgctggcaagctcactatgggacagccgttggtcattctggccccccatgccgtagaggcactagttaagcaaccccctgatcgctggctctccaatgcccggatgacccattaccaagccctgctcctggacacggaccgggtccagttcgggccagtagtggccctaaatccagctacgctgctccctctgcctgaggaggggctgcaacatgactgccttgacatcttggctgaagcccacggaactagatcagatcttacggaccagcccctcccagacgccgaccacacctggtacacggatgggagcagcttcctgcaagaagggcagcgtaaggccggagcagcggtgaccactgagactgaggtaatctgggccagggcattgccagccgggacatcggcccaaagagctgaactgatagcgctcacccaagccctaaagatggcagaaggtaagaagctaaatgtttatactgatagccgttacgcttttgccaccgcccatattcatggagaaatatacagaaggcgcgggttgctcacatcagaaggaaaagagatcaagaacaaggacgagatcttagccctactaaaggctctcttcttgcccaaaagacttagcataattcattgcccgggacatcaaaaaggaaacagcgcagaggccaggggcaaccggatggccgaccaagcggcccgagaagtagccactagagaaactccaggaacttccacacttctgatagaaaactcaaccccctatacccatgaacactttcactatacagtaactgacacaaaggatttgaccaaactaggagccacttatgacagtgcgaagaaatattgggtctatcaaggaaagcctgttatgcctgatcaattcacctttgagttactagactttcttcaccaattgacccacctcagcttctcaaaaacaaaggctctcctagagagaagccccagtccctactacatgctgaaccgggatcgaacactcaaaaatatcactgagacctgcaaagcttgtgcacaagtcaatgccagcaagtctgccgttaagcaaggaactagggtccgcgggcatcggcctggcacacactgggagatcgatttcaccgaggtaaaacctggattgtatggctataagtatcttttagtttttgtagatactttttctggctggatagaagctttcccaactaagaaagaaaccgccaaggtcgtgaccaagaaactgctagaagagatcttccctaggttcggcatgccgcaggtattgggaactgacaatgggcctgccttcgtctccaaggtgagtcagacagtggccgatctgttggggattgattggaaattacattgtgcatacagaccccaaagctcaggtcaggtagaaagaatgaataggaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgaccagagttactaacagcccctctctccaagctcacttacaggctctctacttagtccagcacgaagtttggagaccactggcggcagcttaccaagaacaactggaccggccggtggtgcctcacccttaccgggtcggcgacacagtgtgggtccgccgacatcaaaccaagaacctagaacctcgctggaaaggaccttacacagtcctgctgaccacccccaccgccctcaaagtagacggtatcgcagcttggatacacgcagcccacgtaaaggcggccgacaccgagagtggaccatcctctggacggacatggcgcgttcaacgctctcaaaaccccctcaagataagattaacccgtggaagcccttaatagtcatgggagtcctgttaggagtagggatgacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 6 - DNA, 6180 bp, Artificial Sequence
  phCMV GaLV
cgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataggccgctctagagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggtcgaccgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttgttgtgctgtctcatcattttggcaaagaattctctagagcggtaaaagtcgatggtattgctgcctgggtccatgcttctcacctcaaacctgcaccaccttcggcaccagatgagtcctgggagctggaaaagactgatcatcctcttaagctgcgtattcggcggcggcgggacgagtctgcaaaataagaacccccaccagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaatcgattagttcaatttgttaaagacaggatctcagtagtccaggctttagtcctgactcaacaataccaccagctaaagcctatagagtacgagccatagggcgcctagtgttgacaattaatcatcggcatagtatatcggcatagtataatacgactcactataggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacgcggcccgacgggtccgaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatccctcggagatctgggcccatgcggccgcggatcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccgggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggcttcagatttatcagcaataaaccaccagcccggaagggccgagcgca

SEQ ID NO: 7 - DNA, 21 bp, Artificial Sequence
  oligo 1
ggtcaacttggccatggtggc

SEQ ID NO: 8 - DNA, 21 bp, Artificial Sequence
  oligo 2
cagcccatgaccctcacttgg

SEQ ID NO: 9 - DNA, 21 bp, Artificial Sequence
  oligo 3
ccctactcctaacaggactcc

SEQ ID NO: 10 - DNA, 21 bp, Artificial Sequence
  oligo 4
gtcagagatggctgactgagg

SEQ ID NO: 11 - DNA, 11364 bp, Artificial Sequence
  pRCR GaLV 1
gaattcataccagatcaccgaaaactgtcctccaaatgtgtccccctcacactcccaaattcgcgggcttctgcctcttagaccactctaccctattccccacactcaccggagccaaagccgcggcccttccgtttctttgcttttgaaagaccccacccgtaggtggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtccaaaaatcccgatcgttttggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactagggaggtcagggtcaggagcccccccctgaacccaggataaccctcaaagtcggggggcaacccgtcaccttcctggtagatactggggcccaacactccgtgctgacccaaaatcctggacccctaagtgataagtctgcctgggtccaaggggctactggaggaaagcggtatcgctggaccacggatcgcaaagtacatctagctaccggtaaggtcacccactctttcctccatgtaccagactgtccctatcctctgttaggaagagatttgctgactaaactaaaagcccaaatccactttgagggatcaggagctcaggttatgggaccaatggggcagcccctgcaagtgttgaccctaaatatagaagatgagcatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccttgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggcgtcctaacgcaaaagctgggaccttggcgtcggccggtggcctacctgtctaaaaagctagacccagtggcagctggctggcccccctgcctacggatggtggcagccattgcagttctgacaaaagatgctggcaagctcactatgggacagccgttggtcattctggccccccatgccgtagaggcactagttaagcaaccccctgatcgctggctctccaatgcccggatgacccattaccaagccctgctcctggacacggaccgggtccagttcgggccagtagtggccctaaatccagctacgctgctccctctgcctgaggaggggctgcaacatgactgccttgacatcttggctgaagcccacggaactagatcagatcttacggaccagcccctcccagacgccgaccacacctggtacacggatgggagcagcttcctgcaagaagggcagcgtaaggccggagcagcggtgaccactgagactgaggtaatctgggccagggcattgccagccgggacatcggcccaaagagctgaactgatagcgctcacccaagccctaaagatggcagaaggtaagaagctaaatgtttatactgatagccgttacgcttttgccaccgcccatattcatggagaaatatacagaaggcgcgggttgctcacatcagaaggaaaagagatcaagaacaaggacgagatcttagccctactaaaggctctcttcttgcccaaaagacttagcataattcattgcccgggacatcaaaaaggaaacagcgcagaggccaggggcaaccggatggccgaccaagcggcccgagaagtagccactagagaaactccaggaacttccacacttctgatagaaaactcaaccccctatacccatgaacactttcactatacagtaactgacacaaaggatttgaccaaactaggagccacttatgacagtgcgaagaaatattgggtctatcaaggaaagcctgttatgcctgatcaattcacctttgagttactagactttcttcaccaattgacccacctcagcttctcaaaaacaaaggctctcctagagagaagccccagtccctactacatgctgaaccgggatcgaacactcaaaaatatcactgagacctgcaaagcttgtgcacaagtcaatgccagcaagtctgccgttaagcaaggaactagggtccgcgggcatcggcctggcacacactgggagatcgatttcaccgaggtaaaacctggattgtatggctataagtatcttttagtttttgtagatactttttctggctggatagaagctttcccaactaagaaagaaaccgccaaggtcgtgaccaagaaactgctagaagagatcttccctaggttcggcatgccgcaggtattgggaactgacaatgggcctgccttcgtctccaaggtgagtcagacagtggccgatctgttggggattgattggaaattacattgtgcatacagaccccaaagctcaggtcaggtagaaagaatgaataggaccatcaaggagactttaactaaattaacgcttgcaactggctctagagactgggtgctcctactccccttagccctgtaccgagcccgcaacacgccgggcccccatggcctcaccccatatgagatcttatatggggcacccccgccccttgtaaacttccctgaccctgacatgaccagagttactaacagcccctctctccaagctcacttacaggctctctacttagtccagcacgaagtttggagaccactggcggcagcttaccaagaacaactggaccggccggtggtgcctcacccttaccgggtcggcgacacagtgtgggtccgccgacatcaaaccaagaacctagaacctcgctggaaaggaccttacacagtcctgctgaccacccccaccgccctcaaagtagacggtatcgcagcttggatacacgcagcccacgtaaaggcggccgacaccgagagtggaccatcctctggacggacatggcgcgttcaacgctctcaaaaccccctcaagataagattaacccgtggaagcccttaatagtcatgggagtcctgttaggagtagggcagcccatgaccctcacttggcaggtactgtcccaaactggagacgttgtctgggatacaaaggcagtccagcccccttggacttggtggcccacacttaaacctgatgtatgtgccttggcggctagtcttgagtcctgggatatcccgggaaccgatgtctcgtcctctaaacgagtcagacctccggactcagactatactgccgcttataagcaaatcacctggggagccatagggtgcagctaccctcgggctaggactagaatggcaagctctaccttctacgtatgtccccgggatggccggaccctttcagaagctagaaggtgcggggggctagaatccctatactgtaaagaatgggattgtgagaccacggggaccggttattggctatctaaatcctcaaaagacctcataactgtaaaatgggaccaaaatagcgaatggactcaaaaatttcaacagtgtcaccagaccggctggtgtaacccccttaaaatagatttcacagacaaaggaaaattatccaaggactggataacgggaaaaacctggggattaagattctatgtgtctggacatccaggcgtacagttcaccattcgcttaaaaatcaccaacatgccagctgtggcagtaggtcctgacctcgtccttgtggaacaaggacctcctagaacgtccctcgctctcccacctcctcttcccccaagggaagcgccaccgccatctctccccgactctaactccacagccctggcgactagtgcacaaactcccacggtgagaaaaacaattgttaccctaaacactccgcctcccaccacaggcgacagactttttgatcttgtgcagggggccttcctaaccttaaatgctaccaacccaggggccactgagtcttgctggctttgtttggccatgggccccccttattatgaagcaatagcctcatcaggagaggtcgcctactccaccgaccttgaccggtgccgctgggggacccaaggaaagctcaccctcactgaggtctcaggacacgggttgtgcataggaaaggtgccctttacccatcagcatctctgcaatcagaccctatccatcaattcctccggagaccatcagtatctgctcccctccaaccatagctggtgggcttgcagcactggcctcaccccttgcctctccacctcagtttttaatcagactagagatttctgtatccaggtccagctgattcctcgcatctattactatcctgaagaagttttgttacaggcctatgacaattctcaccccaggactaaaagagaggctgtctcacttaccctagctgttttactggggttgggaatcacggcgggaataggtactggttcaactgccttaattaaaggacctatagacctccagcaaggcctgacaagcctccagatcgccatagatgctgacctccgggccctccaagactcagtcagcaagttagaggactcactgacttccctgtccgaggtagtgctccaaaataggagaggccttgacttgctgtttctaaaagaaggtggcctctgtgcggccctaaaggaagagtgctgtttttacatagaccactcaggtgcagtacgggactccatgaaaaaactcaaagaaaaactggataaaagacagttagagcgccagaaaagccaaaactggtatgaaggatggttcaataactccccttggttcactaccctgctatcaaccatcgctgggcccctattactcctccttctgttgctcatcctcgggccatgcatcatcaatcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 12 - DNA, 64 bp, Artificial Sequence
  oligo 5
gggtcatgggctggtgggggttcttattttgcagactcgtcatccctactcctaacaggactcc

SEQ ID NO: 13 - DNA, 64 bp, Artificial Sequence
  oligo 6
gttaggagtagggatgacgagtctgcaaaataagaacccccaccagcccatgaccctcacttgg

SEQ ID NO: 14 - DNA, 20 bp, Artificial Sequence
  oligo 7
caactggctctagagactgg

SEQ ID NO: 15 - DNA, 20 bp, Artificial Sequence
  oligo 8
cctttcctatgcacaacccg